(19) United States
(12) Patent Application Publication
(10) Pub. No.: US 2004/0202636 A1
(43) Pub. Date: Oct. 14, 2004

(54) Personal care composition containing an antidandruff component and a nonionic surfactant

(76) Inventors: Joseph Robert Kaczvinsky JR., Cincinnati, OH (US); Charles Raymond Degenhardt, Cincinnati, OH (US); David Joseph Eickhoff, Edgewood, KY (US); Celeste Dawn Gale, Hamilton, OH (US); Thomas Larry Dawson, Hamilton, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/412,785

(22) Filed: Apr. 11, 2003

(51) Int. Cl. A61K 7/075; A61K 7/08

(52) U.S. Cl. 424/70.31

(57) ABSTRACT

Personal care compositions that provide improved cleansing and anti-dandruff efficacy. These compositions have from about 5 to about 50 weight percent of a detersive surfactant, from about 0.1 to about 4 weight percent of an anti-dandruff agent, from about 0.1 to about 50 weight percent of an octylphenol ethoxylate or nonylphenol ethoxylate, and at least about 20 weight percent water.

### CROSS REFERENCE TO RELATED APPLICATION(S)

This application is a continuation-in-part of Application Serial No.______ , filed Apr. 4, 2003.

### FIELD

The present invention relates to personal care compositions that provide improved cleansing and anti-dandruff efficacy. These compositions contain detersive surfactants, anti-dandruff agents, octylphenol ethoxylate or nonylphenol ethoxylate, and water.

### BACKGROUND

Anti-dandruff shampoos are well known in the art and are commercially available. Anti-dandruff shampoos typically incorporate an anti-dandruff active and detersive surfactants. Among the preferred types of anti-dandruff agents are particulate, crystalline anti-dandruff agents, such as sulfur, selenium disulfide and heavy metal salts of pyridinethione. Soluble anti-dandruff agents, such as ketoconazole, are also known in the art.

Anti-dandruff shampoos which also provide conditioning benefits are likewise known in the art. For example, U.S. Pat. No. 5,624,666 exemplifies and claims shampoo compositions, containing anionic surfactants, cationic polymers, and zinc pyridinethione as an anti-dandruff agent. U.S. Pat. No. 5,624,666 teaches that conditioning agents such as silicone fluids can optionally be incorporated into the compositions therein. Head & Shoulders® Dandruff Shampoo Plus Conditioner is an example of a marketed product which provides both anti-dandruff and conditioning benefits upon application of the shampoo to hair.

Nevertheless, some consumers desire a personal care composition that provides improved cleansing and anti-dandruff efficacy versus currently marketed products. The present invention addresses this need by providing a personal care composition with improved cleansing and anti-dandruff efficacy.

The present invention further relates to a method for providing improved cleansing and anti-dandruff efficacy by applying an effective amount of the personal care composition to the hair, scalp, or skin in order to provide such benefits.

### SUMMARY

The present invention is directed to a personal care composition comprising:

a) from about 5 to about 50 weight percent of a detersive surfactant,

b) from about 0.1 to about 4 weight percent of an anti-dandruff agent,

c) from about 0.1 to about 50 weight percent of an octylphenol ethoxylate, represented by the formula:

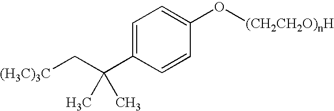

wherein n is a value of from about 3 to about 70, or a nonylphenol ethoxylate, represented by the formula

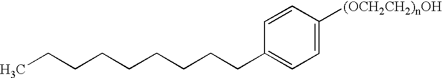

wherein n is a value of from about 4 to about 70, and

d) at least about 20 weight percent of water.

The present invention is further directed to a method of using the composition.

These and other features, aspects, and advantages of the present invention will become evident to those skilled in the art from a reading of the present disclosure.

### DETAILED DESCRIPTION

While the specification concludes with claims that particularly point out and distinctly claim the invention, it is believed the present invention will be better understood from the following description.

The personal care compositions of the present invention include a detersive surfactant, an anti-dandruff agent, an octylphenol ethoxylate or nonylphenol ethoxylate, and water. The personal care compositions of the present invention provide improved cleansing and anti-dandruff efficacy. Such personal care compositions can comprise, consist of, or consist essentially of the essential elements and limitations of the invention described herein, as well as any of the additional or optional ingredients, components or limitations described herein. The components of the personal care compositions, including those that may optionally be added, as well as methods for preparation and methods for use of the personal care composition are, described in detail below.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The term “charge density”, as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “polymer” as used herein shall include materials made by polymerization of one type of monomer or made by two (i.e., copolymers) or more types of monomers.

The term “water soluble” as used herein, means that the polymer is soluble in water in the present composition. In general, the polymer should be soluble at 25° C. at a concentration of 0.1% by weight of the water solvent, preferably at least about 1%, more preferably at least about 5%, even more preferably at least about 15%.

The term “water insoluble” as used herein, means that the polymer is not soluble in water in the present composition. Thus, the polymer is not miscible with water.

### A. Detersive Surfactant

The composition of the present invention includes a detersive surfactant. The detersive surfactant component is included to provide cleaning performance to the composition. The detersive surfactant component in turn comprises anionic detersive surfactant, zwitterionic or amphoteric detersive surfactant, or a combination thereof. Such surfactants should be physically and chemically compatible with the essential components described herein, or should not otherwise unduly impair product stability, aesthetics or performance.

Suitable anionic detersive surfactant components for use in the composition herein include those which are known for use in hair care or other personal care cleansing compositions. The concentration of the anionic surfactant component in the composition should be sufficient to provide the desired cleaning and lather performance, and generally range from about 5% to about 50%, preferably from about 8% to about 30%, more preferably from about 10% to about 25%, even more preferably from about 12% to about 22%, by weight of the composition.

Preferred anionic surfactants suitable for use in the compositions are the alkyl sulfates and alkyl ether sulfates. These materials have the respective formulae ROSO₃M and RO(C₂H₄O)ₓSO₃M, wherein R is alkyl or alkenyl of from about 8 to about 18 carbon atoms, x is an integer having a value of from about 1 to about 10, and M is a cation such as ammonium, alkanolamines, such as triethanolamine, monovalent metals, such as sodium and potassium, and polyvalent metal cations, such as magnesium, and calcium. Preferably, R has from about 8 to about 18 carbon atoms, more preferably from about 10 to about 16 carbon atoms, even more preferably from about 12 to about 14 carbon atoms, in both the alkyl sulfates and alkyl ether sulfates.

The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, and tallow. Lauryl alcohol and straight chain alcohols derived from coconut oil or palm kernel oil are preferred. Such alcohols are optionally reacted with up to about 10, preferably from about 2 to about 5, more preferably about 3, molar proportions of ethylene oxide, and the resulting mixture of molecular species having, for example, an average of 3 moles of ethylene oxide per mole of alcohol, is sulfated and neutralized.

Other suitable anionic detersive surfactants are the water-soluble salts of organic, sulfuric acid reaction products conforming to the formula [R¹—SO₃—M] where R¹ is a straight or branched chain, saturated, aliphatic hydrocarbon radical having from about 8 to about 24, preferably about 10 to about 18, carbon atoms; and M is a cation described hereinbefore.

Still other suitable anionic detersive surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278.

Other anionic detersive surfactants suitable for use in the compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic detersive surfactants include olefin sulfonates having from about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non-limiting example of such an alpha-olefin sulfonate mixture is described in U.S. Pat. 3,332,880.

Another class of anionic detersive surfactants suitable for use in the compositions are the beta-alkyloxy alkane sulfonates. These surfactants conform to the formula (I):

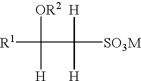

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore.

Preferred anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium cocoyl isethionate and combinations thereof.

Suitable amphoteric or zwitterionic detersive surfactants for use in the composition herein include those that are known for use in hair care or other personal care compositions. The concentration of such amphoteric detersive surfactants preferably ranges from about 0.5% to about 20%, preferably from about 1% to about 10%, by weight of the composition. Non-limiting examples of suitable zwitterionic or amphoteric surfactants are described in U.S. Pat. Nos. 5,104,646 (Bolich Jr. et al.), 5,106,609 (Bolich Jr. et al.).

Amphoteric detersive surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Preferred amphoteric detersive surfactants for use in the present invention include cocoamphoacetate, cocoamphodiacetate, lauroamphoacetate, lauroamphodiacetate, and mixtures thereof.

Zwitterionic detersive surfactants suitable for use in the composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines are preferred.

The compositions of the present invention may further comprise additional surfactants for use in combination with the anionic detersive surfactant component described hereinbefore. Suitable optional surfactants include nonionic and cationic surfactants. Any such surfactant known in the art for use in hair or personal care products may be used, provided that the optional additional surfactant is also chemically and physically compatible with the essential components of the composition, or does not otherwise unduly impair product performance, aesthetics or stability. The concentration of the optional additional surfactants in the composition may vary with the cleansing or lather performance desired, the optional surfactant selected, the desired product concentration, the presence of other components in the composition, and other factors well known in the art.

Non-limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678, 2,658,072; 2,438,091; 2,528,378.

### B. Anti-dandruff Agent

The compositions of the present invention comprise from about 0.1% to about 4%, by weight of the composition, preferably from about 0.1% to about 3%, more preferably from about 0.3% to about 2%, of an anti-dandruff agent suitable for application to the hair or skin. The anti-dandruff agent provides the compositions with anti-microbial activity. The anti-dandruff agent may be particulate or soluble. Suitable, non-limiting examples of anti-dandruff particulates include: pyridinethione salts, azoles, selenium sulfide, particulate sulfur, and mixtures thereof. Preferred are pyridinethione salts. Such anti-dandruff particulate should be physically and chemically compatible with the essential components of the composition and should not otherwise unduly impair product stability, aesthetics or performance.

1. Pyridinethione Salts

Pyridinethione anti-dandruff particulates, especially 1-hydroxy-2-pyridinethione salts, are highly preferred particulate anti-dandruff agents for use in compositions of the present invention. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form, wherein the particles have an average size of up to about 20μ, preferably up to about 5μ, more preferably up to about 2.5μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. It is contemplated that when ZPT is used as the anti-dandruff particulate in the compositions herein, that the growth or re-growth of hair may be stimulated or regulated, or both, or that hair loss may be reduced or inhibited, or that hair may appear thicker or fuller. 2. Other Anti-Microbial Actives

The present invention may comprise one or more anti-fungal or anti-microbial actives. Suitable anti-microbial actives include coal tar, sulfur, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP- 100, azelaic acid, lyticase, iodopropynyl butylcarbamate (IPBC), isothiazalinones such as octyl isothiazalinone and azoles, and combinations thereof. Additional anti-microbial actives of the present invention may include extracts of melaleuca (tea tree) and charcoal. Preferred anti-microbials include itraconazole, ketoconazole, selenium sulphide and coal tar.

The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, and mixtures thereof.

3. Azoles

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. Especially preferred herein is ketoconazole.

4. Selenium Sulfide

Selenium sulfide is a particulate anti-dandruff agent suitable for use in the compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

5. Sulfur

Sulfur may also be used as a particulate anti-dandruff agent in the compositions of the present invention. Effective concentrations of the particulate sulfur are typically from about 1% to about 4%, by weight of the composition, preferably from about 2% to about 4%.

6. Keratolytic Agents

The present invention may further comprise one or more keratolytic agents such as Salicylic Acid.

### C. TRITON X series and TERGITOL NP series Surfactants

The compositions of the present invention comprise at least one octylphenol ethoxylate, represented by the following formula (II)

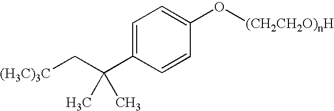

wherein, n is a value of from about 3 to about 70, preferably from about 5 to about 20, more preferably from about 5 to about 10, or a nonylphenol ethoxylate, represented by the following formula (III)

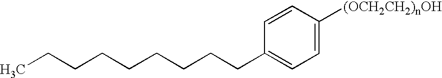

wherein, n is a value of from about 4 to about 70, preferably from about 4 to about 30, more preferably from about 7 to about 15.

One common tradename for the octylphenol ethoxylates is TRITON X series, and a common tradename for the nonylphenol ethoxylates is TERGITOL NP series. The TRITON X series or TERGITOL NP series surfactant is present in the personal care composition in an amount preferably greater than 0.1%, more preferably greater than 1%, still more preferably greater than 2%. Preferably the amount of surfactant less than about 50%, more preferably less than about 20%, and still more preferably less than about 10%.

Specific examples of TRITON X series and TERGITOL NP series surfactants that are useful in the present invention include TRITON X-100, TRITON X-45, TERGITOL NP- 10, and TERGITOL NP-30, (all commercially available from Dow Chemical).

### D. Water

The compositions of the present invention comprise from about 20% to about 94.75%, by weight of the composition, preferably from about 50% to about 94.75%, more preferably from about 60% to about 85%, of water.

### E. Optional Components

The compositions of the present invention may further comprise one or more optional components known for use in hair care or personal care products, provided that the optional components are physically and chemically compatible with the essential components described herein, or do not otherwise unduly impair product stability, aesthetics or performance. Individual concentrations of such optional components may range from about 0.001% to about 10% by weight of the compositions.

Non-limiting examples of optional components for use in the composition include a deposition aid, cationic polymers, nonionic polymers, dispersed particles, conditioning agents (silicones and organic conditioning oils), humectant, suspending agent, additional anti-dandruff actives, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

1. Deposition Aid

The personal care compositions of the present invention may include a deposition aid. The deposition aid is included to effectively enhance deposition of the personal care composition components. The deposition aid can comprise any material that enhances the deposition of the personal care composition components onto the hair, scalp, or skin. Preferably, the deposition aids are cationic polymers. The concentration of the deposition aid in the personal care composition should be sufficient to effectively enhance the deposition of the components and typically range from about 0.05% to about 5%, preferably from about 0.075% to about 2.5%, more preferably from about 0.1% to about 1.0%, by weight of the personal care composition.

2. Cationic Polymers

The compositions of the present invention may contain a cationic polymer. Concentrations of the cationic polymer in the composition typically range from about 0.05% to about 3%, preferably from about 0.075% to about 2.0%, more preferably from about 0.1% to about 1.0%, by weight of the composition. Preferred cationic polymers will have cationic charge densities of at least about 0.9 meq/gm, preferably at least about 1.2 meq/gm, more preferably at least about 1.5 meq/gm, but also preferably less than about 7 meq/gm, more preferably less than about 5 meq/gm. The pH of intended use of the composition will generally range from about pH 3 to about pH 9, preferably between about pH 4 and about pH 8. The average molecular weight of such suitable cationic polymers will generally be between about 10,000 and 10 million, preferably between about 50,000 and about 5 million, more preferably between about 100,000 and about 3 million.

Suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non-limiting examples of such counterions include halides (e.g., chloride, fluoride, bromide, iodide), sulfate and methylsulfate.

Non-limiting examples of cationic polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Non-limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). Preferred cationic substituted monomers are the cationic substituted dialkylaminoalkyl acrylamides, dialkylaminoalkyl methacrylamides, and combinations thereof. These preferred monomers conform the to the formula (IV):

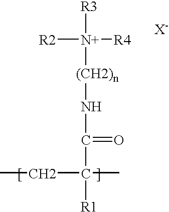

wherein R¹ is hydrogen, methyl or ethyl; each of R², R3 and R4 are independently hydrogen or a short chain alkyl having from about 1 to about 8 carbon atoms, preferably from about 1 to about 5 carbon atoms, more preferably from about 1 to about 2 carbon atoms; n is an integer having a value of from about 1 to about 8, preferably from about 1 to about 4; and X is a counterion. The nitrogen attached to R², R³ and R⁴ may be a protonated amine (primary, secondary or tertiary), but is preferably a quaternary ammonium wherein each of R², R³ and R⁴ are alkyl groups a non-limiting example of which is polymethyacrylamidopropyl trimonium chloride, available under the trade name Polycare 133, from Rhone-Poulenc, Cranberry, N.J., U.S.A. Also preferred are copolymers of the above cationic monomer with nonionic monomers such that the charge density of the total copolymer is from about 2.0 to about 4.5 meq/gm.

Other suitable cationic polymers for use in the composition include polysaccharide polymers, such as cationic cellulose derivatives and cationic starch derivatives. Suitable cationic polysaccharide polymers include those which conform to the formula (V):

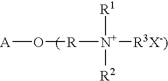

wherein A is an anhydroglucose residual group, such as a starch or cellulose anhydroglucose residual; R is an alkylene oxyalkylene, polyoxyalkylene, or hydroxyalkylene group, or combination thereof; R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less; and X is an anionic counterion as described in hereinbefore.

Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum derivatives, such as guar hydroxypropyltrimonium chloride, specific examples of which include the Jaguar series commercially avaialable from Rhone-Poulenc Incorporated and the N-Hance series commercially available from Aqualon Division of Hercules, Inc. Other suitable cationic polymers include quaternary nitrogen-containing cellulose ethers, some examples of which are described in U.S. Pat. No. 3,962,418. Other suitable cationic polymers include copolymers of etherified cellulose, guar and starch, some examples of which are described in U.S. Pat. No. 3,958,581. When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

3. Nonionic polymers

Polyalkylene glycols having a molecular weight of more than about 1000 are useful herein. Useful are those having the following general formula (VI):

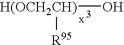

wherein R⁹⁵ is selected from the group consisting of H, methyl, and mixtures thereof. Polyethylene glycol polymers useful herein are PEG-2M (also known as Polyox WSR® N-10, which is available from Union Carbide and as PEG-2,000); PEG-5M (also known as Polyox WSR®N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

4. Dispersed Particles

The composition of the present invention may include dispersed particles. The compositions of the present invention, may include at least 0.025% by weight of the dispersed particles, more preferably at least 0.05%, still more preferably at least 0. %, even more preferably at least 0.25%, and yet more preferably at least 0.5% by weight of the dispersed particles. In the compositions of the present invention, it is preferable to incorporate no more than about 20% by weight of the dispersed particles, more preferably no more than about 10%, still more preferably no more than 5%, even more preferably no more than 3%, and yet more preferably no more than 2% by weight of the dispersed particles.

5. Conditioning Agents

Conditioning agents include any material which is used to give a particular conditioning benefit to hair and/or skin. The conditioning agents useful in the compositions of the present invention typically comprise a water insoluble, water dispersible, non-volatile, liquid that forms emulsified, liquid particles or are solubilized by the surfactant micelles, in the anionic detersive surfactant component (described above). Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein.

a. Silicones

The conditioning agent of the compositions of the present invention is preferably an insoluble silicone conditioning agent. The silicone conditioning agent particles may comprise volatile silicone, non-volatile silicone, or combinations thereof. Preferred are non-volatile silicone conditioning agents. If volatile silicones are present, they will typically be incidental to their use as a solvent or carrier for commercially available forms of non-volatile silicone material ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The concentration of the silicone conditioning agent typically ranges from about 0.01% to about 10%, by weight of the composition, preferably from about 0.1% to about 8%, more preferably from about 0.1% to about 5%, more preferably from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the compositions of the present invention preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, more preferably from about 100,000 to about 1,500,000 csk.

The dispersed silicone conditioning agent particles typically have a volume average particle diameter ranging from about 0.01 μm to about 50 μm. For small particle application to hair, the volume average particle diameters typically range from about 0.01 μm to about 4 μm, preferably from about 0.01 μm to about 2 μm, more preferably from about 0.01 μm to about 0.5 μm. For larger particle application to hair, the volume average particle diameters typically range from about 5 μm to about 125 μm, preferably from about 10 μm to about 90 μm, more preferably from about 15 μm to about 70 μm, more preferably from about 20 μm to about 50 μm.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

i. Silicone Oils

Silicone fluids include silicone oils, which are flowable silicone materials having a viscosity, as measured at 25° C., less than 1,000,000 csk, preferably from about 5 csk to about 1,000,000 csk, more preferably from about 100 csk to about 600,000 csk. Suitable silicone oils for use in the compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following formula (VII):

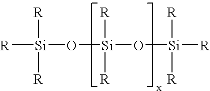

wherein R is aliphatic, preferably alkyl or alkenyl, or aryl, R can be substituted or unsubstituted, and x is an integer from 1 to about 8,000. Suitable R groups for use in the compositions of the present invention include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Preferred alkyl and alkenyl substituents are C₁ to C₅ alkyls and alkenyls, more preferably from C₁ to C₄, more preferably from C₁ to C₂. The aliphatic portions of other alkyl-, alkenyl-, or alkynyl-containing groups (such as alkoxy, alkaryl, and alkamino) can be straight or branched chains, and are preferably from C₁ to C₅, more preferably from C₁ to C₄, even more preferably from C₁ to C₃, more preferably from C₁ to C₂. As discussed above, the R substituents can also contain amino functionalities (e.g. alkamino groups), which can be primary, secondary or tertiary amines or quaternary ammonium. These include mono-, di- and tri- alkylamino and alkoxyamino groups, wherein the aliphatic portion chain length is preferably as described herein.

ii. Amino and Cationic Silicones

Cationic silicone fluids suitable for use in the compositions of the present invention include, but are not limited to, those which conform to the general formula (VIII):

(R₁)ₐG₃₋ₐ—Si—(—OSiG₂)ₙ—(—OSiG_{b}(R₁)_{2-b)m}—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 0; b is 0 or 1, preferably 1; n is a number from 0 to 1,999, preferably from 49 to 499; m is an integer from 1 to 2,000, preferably from 1 to 10; the sum of n and m is a number from 1 to 2,000, preferably from 50 to 500; R₁ is a monovalent radical conforming to the general formula CqH_{2q}L, wherein q is an integer having a value from 2 to 8 and L is selected from the following groups:

—N(R₂)CH₂—CH₂—N(R₂)₂

—N(R₂)₂

—N(R₂)₃A⁻

—N(R₂)CH₂—CH₂—NR₂H₂A⁻

wherein R₂ is hydrogen, phenyl, benzyl, or a saturated hydrocarbon radical, preferably an alkyl radical from about C₁ to about C₂₀, and A⁻ is a halide ion.

iii. Silicone Gums

Other silicone fluids suitable for use in the compositions of the present invention are the insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76. Specific non-limiting examples of silicone gums for use in the compositions of the present invention include polydimethylsiloxane, (polydimethylsiloxane) (methylvinylsiloxane) copolymer, poly(dimethylsiloxane) (diphenyl siloxane)(methyl-vinylsiloxane) copolymer and mixtures thereof.

iv. High refractive Index Silicones

Other non-volatile, insoluble silicone fluid conditioning agents that are suitable for use in the compositions of the present invention are those known as “high refractive index silicones,” having a refractive index of at least about 1.46, preferably at least about 1.48, more preferably at least about 1.52, more preferably at least about 1.55. The refractive index of the polysiloxane fluid will generally be less than about 1.70, typically less than about 1.60. In this context, polysiloxane “fluid” includes oils as well as gums.

The high refractive index polysiloxane fluid includes those represented by general formula (VII) above, as well as cyclic polysiloxanes such as those represented by formula (IX) below:

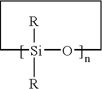

wherein R is as defined above, and n is a number from about 3 to about 7, preferably from about 3 to about 5.

The high refractive index polysiloxane fluids contain an amount of aryl-containing R substituents sufficient to increase the refractive index to the desired level, which is described herein. Additionally, R and n must be selected so that the material is non-volatile.

Aryl-containing substituents include those which contain alicyclic and heterocyclic five and six member aryl rings and those which contain fused five or six member rings. The aryl rings themselves can be substituted or unsubstituted.

Preferred high refractive index polysiloxane fluids have a combination of phenyl or phenyl derivative substituents (more preferably phenyl), with alkyl substituents, preferably C₁-C₄ alkyl (more preferably methyl), hydroxy, or C₁-C₄ alkylamino (especially —R¹NHR²NH2 wherein each R¹ and R² independently is a C₁-C₃ alkyl, alkenyl, and/or alkoxy).

Silicone fluids suitable for use in the compositions of the present invention are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

v. Silicone Resins

Silicone resins may be included in the silicone conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence.

Preferred silicone resins for use in the compositions of the present invention include, but are not limited to MQ, MT, MTQ, MDT and MDTQ resins. Methyl is a preferred silicone substituent. Especially preferred silicone resins are MQ resins, wherein the M:Q ratio is from about 0.5:1.0 to about 1.5:1.0 and the average molecular weight of the silicone resin is from about 1000 to about 10,000.

b. Organic Conditioning Oils

The conditioning component of the compositions of the present invention may also comprise from about 0.05% to about 3%, by weight of the composition, preferably from about 0.08% to about 1.5%, more preferably from about 0.1% to about 1%, of at least one organic conditioning oil as the conditioning agent, either alone or in combination with other conditioning agents, such as the silicones (described above).

i. Hydrocarbon Oils

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2, 2, 4, 4, 6, 6, 8, 8-dimethyl-10-methylundecane and 2, 2, 4, 4, 6, 6-dimethyl-8-methylnonane, available from Permethyl Corporation. Hydrocarbon polymers such as polybutene and polydecene are preferred. A preferred hydrocarbon polymer is polybutene, such as the copolymer of isobutylene and butene. A commercially available material of this type is L-14 polybutene from Amoco Chemical Corporation.

ii. Polyolefins

Organic conditioning oils for use in the compositions of the present invention can also include liquid polyolefins, more preferably liquid poly-α-olefins, more preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. Preferred hydrogenated α-olefin monomers include, but are not limited to: 1-hexene to 1-hexadecenes, 1-octene to 1-tetradecene, and mixtures thereof.

iii. Fatty Esters

Other suitable organic conditioning oils for use as the conditioning agent in the compositions of the present invention include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to: isopropyl isostearate, hexyl laurate, isohexyl laurate, isohexyl palmitate, isopropyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are mono-carboxylic acid esters of the general formula R′COOR, wherein R′ and R are alkyl or alkenyl radicals, and the sum of carbon atoms in R′ and R is at least 10, preferably at least 22.

Still other fatty esters suitable for use in the compositions of the present invention are di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific non-limiting examples of di- and tri- alkyl and alkenyl esters of carboxylic acids include isocetyl stearyol stearate, diisopropyl adipate, and tristearyl citrate.

Other fatty esters suitable for use in the compositions of the present invention are those known as polyhydric alcohol esters. Such polyhydric alcohol esters include alkylene glycol esters, such as ethylene glycol mono- and di-fatty acid esters, diethylene glycol mono- and di-fatty acid esters, polyethylene glycol mono- and di-fatty acid esters, propylene glycol mono- and di- fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Still other fatty esters suitable for use in the compositions of the present invention are glycerides, including, but not limited to, mono-, di-, and tri-glycerides, preferably di- and tri-glycerides, more preferably triglycerides. For use in the compositions described herein, the glycerides are preferably the mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Other fatty esters suitable for use in the compositions of the present invention are water insoluble synthetic fatty esters.

Specific non-limiting examples of suitable synthetic fatty esters for use in the compositions of the present invention include: P-43 (C₈-C₁₀ triester of trimethylolpropane), MCP-684 (tetraester of 3,3 diethanol-1,5 pentadiol), MCP 121 (C₈-C₁₀ diester of adipic acid), all of which are available from Mobil Chemical Company.

c. Other Conditioning Agents

Also suitable for use in the compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586 (Clairol), 4,507,280 (Clairol), 4,663,158 (Clairol), 4,197,865 (L'Oreal), 4,217, 914 (L'Oreal), 4,381,919 (L'Oreal), and 4,422, 853 (L'Oreal).

6. Humectant

The compositions of the present invention may contain a humectant. The humectants herein are selected from the group consisting of polyhydric alcohols, water soluble alkoxylated nonionic polymers, and mixtures thereof. The humectants, when used herein, are preferably used at levels by weight of the composition of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1, 2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof.

7. Suspending Agent

The compositions of the present invention may further comprise a suspending agent at concentrations effective for suspending water-insoluble material in dispersed form in the compositions or for modifying the viscosity of the composition. Such concentrations range from about 0.1% to about 10%, preferably from about 0.3% to about 5.0%, by weight of the compositions.

Suitable suspending agents include crystalline suspending agents that can be categorized as acyl derivatives, long chain amine oxides, or combinations thereof. These suspending agents are described in U.S. Pat. No. 4,741,855.

8. Other Optional Components

The compositions of the present invention may contain also vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

The compositions of the present invention may also contain pigment materials such as nitroso, monoazo, diazo, carotenoid, triphenyl methanes, triaryl methanes, xanthenes, quinolines, oxazines, azines, anthraquinones, indigoids, thionindigoids, quinacridones, phthalocyianines, botanicals, and natural colors including water soluble dye components. The compositions of the present invention may also contain chelating agents.

### METHODS OF MANUFACTURE

The compositions in general can be made by mixing most of the materials together at elevated temperatures, e.g. about 72° C. If no insoluble materials exist, the composition can be made at ambient temperatures. A triblender and/or mill can be used in each step, if necessary, to disperse the materials. Preferably, the heated solution contains at least a portion of the surfactant and any solids requiring melting or solubilization (e.g. Ethylene glycol & Cocamide MEA). The dimethicone, if any, is first mixed together with surfactant prior to being added to the remainder of the product. After all ingredients are added, they are thoroughly heated & mixed and then pumped through a high shear mixer and cooled through a heat exchanger to around 33° C. Also, alternatively, a portion of the soluble and liquid materials can be added after the mix is cooled to 33° C. Zinc pyrithione or selenium sulfide are preferably added after cooling and mixed in thoroughly.

### METHODS OF USE

The composition compositions of the present invention are used in a conventional manner for cleansing and conditioning the hair or skin. They are particularly used in a conventional manner for treating the condition commonly known as dandruff. An effective amount of the composition for cleansing and conditioning the hair or skin is applied to hair, or other region of the body, that has preferably been wetted, generally with water, and then the composition is rinsed off. Effective amounts typically range from about 1 g to about 50 g, preferably from about 1 g to about 20 g. Application to the hair typically includes working the composition through the hair such that most or all of the hair is contacted with the composition.

This method for providing improved cleansing and anti-dandruff efficacy comprises the steps of: (a) wetting the hair or skin with water, (b) applying an effective amount of the personal care composition to the hair or skin, and (c) rinsing the personal care composition from the hair or skin using water. These steps may be repeated as many times as desired to achieve the cleansing, conditioning, and anti-dandruff benefits sought.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified. As used herein, “minors” refers to those optional components such as preservatives, viscosity modifiers, pH modifiers, fragrances, foam boosters, and the like. As is apparent to one of ordinary skill in the art, the selection of these minors will vary depending on the physical and chemical characteristics of the particular ingredients selected to make the present invention as described herein.

| | Example Number | | | | | | | | |
|---|---|---|---|---|---|---|---|---|---|
| | A | B | C | D | E | F | G | H | I |
| Ammonium Laureth Sulfate | 7.5 | 10 | 10 | 10 | — | 10 | 10 | — | 14.13 |
| Ammonium Lauryl Sulfate | 5.5 | 6 | 6 | 6 | — | 6 | 6 | — | 3.15 |
| Ammonium Xylenesulfonat | — | — | — | — | — | — | — | — | 0.46 |
| Sodium Laureth Sulfate | — | — | — | — | 10 | — | — | 10 | — |
| Sodium Lauryl Sulfate | — | — | — | — | 6 | — | — | 6 | — |
| TRITON X-100 | 5 | 5 | 5 | 5 | 5 | 5 | 5 | 5 | 5 |
| Polyquaternium-10¹ | 0.35 | — | 0.5 | 0.1 | 0.5 | — | 0.5 | — | 0.35 |
| Guar | 0.25 | — | — | 0.4 | — | 0.5 | — | 0.5 | — |
| Hydroxypropyltrimonium | | | | | | | | | |
| Chloride² | | | | | | | | | |
| Tricetylmonium Chloride | — | — | — | — | — | — | — | — | 0.5 |
| PEG7M³ | 0.05 | — | 0.1 | 0.1 | 0.1 | — | — | — | — |
| Zinc Pyrithione⁴ | 1 | 1 | 1 | 1 | 1 | 1 | 1 | 1 | — |
| Zinc Oxide | | — | — | — | — | — | — | 1.2 | — |
| Selenium Sulfide | | — | — | — | — | — | — | — | 1.0 |
| 1-decene homopolymer⁵ | 0.4 | — | 0.3 | 0.4 | 0.3 | 0.4 | 0.4 | 0.4 | — |
| Trimethylpropane Capyl | 0.2 | — | 0.1 | 0.1 | 0.1 | 0.1 | 0.1 | 0.1 | — |
| Caprylate⁶ | | | | | | | | | |
| Dimethicone⁷ | 3.25 | — | 1.3 | 1.3 | 1.3 | 2.5 | 2.2 | 2.2 | 0.63 |
| Ethylene Glycol Distearate | 1.0 | 1.0 | 1.5 | 1.5 | 1.5 | 1.5 | 1.5 | 1.5 | 3.0 |
| Cocamide MEA | 0.6 | 0.6 | 0.8 | 0.8 | 0.8 | 0.8 | 1.0 | 0.8 | 3.0 |
| Cetyl Alcohol | 0.6 | — | 0.9 | 0.9 | 0.9 | 0.9 | 0.9 | 0.9 | 0.42 |
| DMDM Hydantoin | — | — | — | — | — | — | — | — | 0.4 |
| Sodium Chloride | — | — | — | — | — | — | — | — | 0.3 |
| Stearyl Alcohol | — | — | — | — | — | — | — | — | 0.2 |
| Hydroxypropyl | — | — | — | — | — | — | — | — | 0.02 |
| Methylcellulose | | | | | | | | | |
| Water and minors | quantity sufficient | | | | | | | | |
| | Example Number | | | | | | | | |
| | J | K | L | M | N | O | P | Q | R |
| Ammonium Laureth Sulfate | 7.5 | 10 | 10 | 10 | — | 10 | 10 | — | 14.13 |
| Ammonium Lauryl Sulfate | 5.5 | 6 | 6 | 6 | — | 6 | 6 | — | 3.15 |
| Ammonium Xylenesulfonat | — | — | — | — | — | — | — | — | 0.46 |
| Sodium Laureth Sulfate | — | — | — | — | 10 | — | — | 10 | — |
| Sodium Lauryl Sulfate | — | — | — | — | 6 | — | — | 6 | — |
| TRITON X-100 | 3 | 3 | 3 | 3 | 3 | 3 | 3 | 3 | 3 |
| Polyquaternium-10¹ | 0.35 | — | 0.5 | 0.1 | 0.5 | — | 0.5 | — | 0.35 |
| Guar | 0.25 | — | — | 0.4 | — | 0.5 | — | 0.5 | — |
| Hydroxypropyltrimonium | | | | | | | | | |
| Chloride² | | | | | | | | | |
| Tricetylmonium Chloride | — | — | — | — | — | — | — | — | 0.5 |
| PEG7M³ | 0.05 | — | 0.1 | 0.1 | 0.1 | — | — | — | — |
| Zinc Pyrithione⁴ | 1 | 1 | 1 | 1 | 1 | 1 | 1 | 1 | — |
| Zinc Oxide | | — | — | — | — | — | — | 1.2 | — |
| Selenium Sulfide | | — | — | — | — | — | — | — | 1.0 |
| 1-decene homopolymer⁵ | 0.4 | — | 0.3 | 0.4 | 0.3 | 0.4 | 0.4 | 0.4 | — |
| Trimethylpropane Capyl | 0.2 | — | 0.1 | 0.1 | 0.1 | 0.1 | 0.1 | 0.1 | — |
| Caprylate⁶ | | | | | | | | | |
| Dimethicone⁷ | 3.25 | — | 1.3 | 1.3 | 1.3 | 2.5 | 2.2 | 2.2 | 0.63 |
| Ethylene Glycol Distearate | 1.0 | 1.0 | 1.5 | 1.5 | 1.5 | 1.5 | 1.5 | 1.5 | 3.0 |
| Cocamide MEA | 0.6 | 0.6 | 0.8 | 0.8 | 0.8 | 0.8 | 1.0 | 0.8 | 3.0 |
| Cetyl Alcohol | 0.6 | — | 0.9 | 0.9 | 0.9 | 0.9 | 0.9 | 0.9 | 0.42 |
| DMDM Hydantoin | — | — | — | — | — | — | — | — | 0.4 |
| Sodium Chloride | — | — | — | — | — | — | — | — | 0.3 |
| Stearyl Alcohol | — | — | — | — | — | — | — | — | 0.2 |
| Hydroxypropyl | — | — | — | — | — | — | — | — | 0.02 |
| Methylcellulose | | | | | | | | | |
| Water and minors | quantity sufficient | | | | | | | | |
| | Example Number | | | | | | | | |
| | S | T | U | V | W | X | Y | Z | AA |
| Ammonium Laureth Sulfate | 7.5 | 10 | 10 | 10 | — | 10 | 10 | — | 14.13 |
| Ammonium Lauryl Sulfate | 5.5 | 6 | 6 | 6 | — | 6 | 6 | — | 3.15 |
| Ammonium Xylenesulfonat | — | — | — | — | — | — | — | — | 0.46 |
| Sodium Laureth Sulfate | — | — | — | — | 10 | — | — | 10 | — |
| Sodium Lauryl Sulfate | — | — | — | — | 6 | — | — | 6 | — |
| TRITON X-45 | 3 | 3 | 3 | 3 | 3 | 3 | 3 | 3 | 3 |
| Polyquaternium-10¹ | 0.35 | — | 0.5 | 0.1 | 0.5 | — | 0.5 | — | 0.35 |
| Guar | 0.25 | — | — | 0.4 | — | 0.5 | — | 0.5 | — |
| Hydroxypropyltrimonium | | | | | | | | | |
| Chloride² | | | | | | | | | |
| Tricetylmonium Chloride | — | — | — | — | — | — | — | — | 0.5 |
| PEG7M³ | 0.05 | — | 0.1 | 0.1 | 0.1 | — | — | — | — |
| Zinc Pyrithione⁴ | 1 | 1 | 1 | 1 | 1 | 1 | 1 | 1 | — |
| Zinc Oxide | | — | — | — | — | — | — | 1.2 | — |
| Selenium Sulfide | | — | — | — | — | — | — | — | 1.0 |
| 1-decene homopolymer⁵ | 0.4 | — | 0.3 | 0.4 | 0.3 | 0.4 | 0.4 | 0.4 | — |
| Trimethylpropane Capyl | 0.2 | — | 0.1 | 0.1 | 0.1 | 0.1 | 0.1 | 0.1 | — |
| Caprylate⁶ | | | | | | | | | |
| Dimethicone⁷ | 3.25 | — | 1.3 | 1.3 | 1.3 | 2.5 | 2.2 | 2.2 | 0.63 |
| Ethylene Glycol Distearate | 1.0 | 1.0 | 1.5 | 1.5 | 1.5 | 1.5 | 1.5 | 1.5 | 3.0 |
| Cocamide MEA | 0.6 | 0.6 | 0.8 | 0.8 | 0.8 | 0.8 | 1.0 | 0.8 | 3.0 |
| Cetyl Alcohol | 0.6 | — | 0.9 | 0.9 | 0.9 | 0.9 | 0.9 | 0.9 | 0.42 |
| DMDM Hydantoin | — | — | — | — | — | — | — | — | 0.4 |
| Sodium Chloride | — | — | — | — | — | — | — | — | 0.3 |
| Stearyl Alcohol | — | — | — | — | — | — | — | — | 0.2 |
| Hydroxypropyl | — | — | — | — | — | — | — | — | 0.02 |
| Methylcellulose | | | | | | | | | |
| Water and minors | quantity sufficient | | | | | | | | |
| | Example Number | | | | | | | | |
| | BB | CC | DD | EE | FF | GG | HH | II | JJ |
| Ammonium Laureth Sulfate | 7.5 | 10 | 10 | 10 | — | 10 | 10 | — | 14.13 |
| Ammonium Lauryl Sulfate | 5.5 | 6 | 6 | 6 | — | 6 | 6 | — | 3.15 |
| Ammonium Xylenesulfonat | — | — | — | — | — | — | — | — | 0.46 |
| Sodium Laureth Sulfate | — | — | — | — | 10 | — | — | 10 | — |
| Sodium Lauryl Sulfate | — | — | — | — | 6 | — | — | 6 | — |
| TERGITOL NP-10 | 3 | 3 | 3 | 3 | 3 | 3 | 3 | 3 | 3 |
| Polyquaternium-10¹ | 0.35 | — | 0.5 | 0.1 | 0.5 | — | 0.5 | — | 0.35 |
| Guar | 0.25 | — | — | 0.4 | — | 0.5 | — | 0.5 | — |
| Hydroxypropyltrimonium | | | | | | | | | |
| Chloride² | | | | | | | | | |
| Tricetylmonium Chloride | — | — | — | — | — | — | — | — | 0.5 |
| PEG7M³ | 0.05 | — | 0.1 | 0.1 | 0.1 | — | — | — | — |
| Zinc Pyrithione⁴ | 1 | 1 | 1 | 1 | 1 | 1 | 1 | 1 | — |
| Zinc Oxide | | — | — | — | — | — | — | 1.2 | — |
| Selenium Sulfide | | — | — | — | — | — | — | — | 1.0 |
| 1-decene homopolymer⁵ | 0.4 | — | 0.3 | 0.4 | 0.3 | 0.4 | 0.4 | 0.4 | — |
| Trimethylpropane Capyl | 0.2 | — | 0.1 | 0.1 | 0.1 | 0.1 | 0.1 | 0.1 | — |
| Caprylate⁶ | | | | | | | | | |
| Dimethicone⁷ | 3.25 | — | 1.3 | 1.3 | 1.3 | 2.5 | 2.2 | 2.2 | 0.63 |
| Ethylene Glycol Distearate | 1.0 | 1.0 | 1.5 | 1.5 | 1.5 | 1.5 | 1.5 | 1.5 | 3.0 |
| Cocamide MEA | 0.6 | 0.6 | 0.8 | 0.8 | 0.8 | 0.8 | 1.0 | 0.8 | 3.0 |
| Cetyl Alcohol | 0.6 | — | 0.9 | 0.9 | 0.9 | 0.9 | 0.9 | 0.9 | 0.42 |
| DMDM Hydantoin | — | — | — | — | — | — | — | — | 0.4 |
| Sodium Chloride | — | — | — | — | — | — | — | — | 0.3 |
| Stearyl Alcohol | — | — | — | — | — | — | — | — | 0.2 |
| Hydroxypropyl | — | — | — | — | — | — | — | — | 0.02 |
| Methylcellulose | | | | | | | | | |
| Water and minors | quantity sufficient | | | | | | | | |
| | Example Number | | | | | | | | |
| | KK | LL | M | NN | OO | PP | QQ | RR | SS |
| Ammonium Laureth Sulfate | 7.5 | 10 | 10 | 10 | — | 10 | 10 | — | 14.13 |
| Ammonium Lauryl Sulfate | 5.5 | 6 | 6 | 6 | — | 6 | 6 | — | 3.15 |
| Ammonium Xylenesulfonat | — | — | — | — | — | — | — | — | 0.46 |
| Sodium Laureth Sulfate | — | — | — | — | 10 | — | — | 10 | — |
| Sodium Lauryl Sulfate | — | — | — | — | 6 | — | — | 6 | — |
| TERGITOL NP-10 | 5 | 5 | 5 | 5 | 5 | 5 | 5 | 5 | 5 |
| Polyquaternium-10¹ | 0.35 | — | 0.5 | 0.1 | 0.5 | — | 0.5 | — | 0.35 |
| Guar | 0.25 | — | — | 0.4 | — | 0.5 | — | 0.5 | — |
| Hydroxypropyltrimonium | | | | | | | | | |
| Chloride² | | | | | | | | | |
| Tricetylmonium Chloride | — | — | — | — | — | — | — | — | 0.5 |
| PEG7M³ | 0.05 | — | 0.1 | 0.1 | 0.1 | — | — | — | — |
| Zinc Pyrithione⁴ | 1 | 1 | 1 | 1 | 1 | 1 | 1 | 1 | — |
| Zinc Oxide | | — | — | — | — | — | — | 1.2 | — |
| Selenium Sulfide | | — | — | — | — | — | — | — | 1.0 |
| 1-decene homopolymer⁵ | 0.4 | — | 0.3 | 0.4 | 0.3 | 0.4 | 0.4 | 0.4 | — |
| Trimethylpropane Capyl | 0.2 | — | 0.1 | 0.1 | 0.1 | 0.1 | 0.1 | 0.1 | — |
| Caprylate⁶ | | | | | | | | | |
| Dimethicone⁷ | 3.25 | — | 1.3 | 1.3 | 1.3 | 2.5 | 2.2 | 2.2 | 0.63 |
| Ethylene Glycol Distearate | 1.0 | 1.0 | 1.5 | 1.5 | 1.5 | 1.5 | 1.5 | 1.5 | 3.0 |
| Cocamide MEA | 0.6 | 0.6 | 0.8 | 0.8 | 0.8 | 0.8 | 1.0 | 0.8 | 3.0 |
| Cetyl Alcohol | 0.6 | — | 0.9 | 0.9 | 0.9 | 0.9 | 0.9 | 0.9 | 0.42 |
| DMDM Hydantoin | — | — | — | — | — | — | — | — | 0.4 |
| Sodium Chloride | — | — | — | — | — | — | — | — | 0.3 |
| Stearyl Alcohol | — | — | — | — | — | — | — | — | 0.2 |
| Hydroxypropyl | — | — | — | — | — | — | — | — | 0.02 |
| Methylcellulose | | | | | | | | | |
| Water and minors | quantity sufficient | | | | | | | | |
| | Example Number | | | | | | | | |
| | TT | UU | VV | W | XX | YY | ZZ | AAA | BBB |
| Ammonium Laureth Sulfate | 7.5 | 10 | 10 | 10 | — | 10 | 10 | — | 14.13 |
| Ammonium Lauryl Sulfate | 5.5 | 6 | 6 | 6 | — | 6 | 6 | — | 3.15 |
| Ammonium Xylenesulfonat | — | — | — | — | — | — | — | — | 0.46 |
| Sodium Laureth Sulfate | — | — | — | — | 10 | — | — | 10 | — |
| Sodium Lauryl Sulfate | — | — | — | — | 6 | — | — | 6 | — |
| TERGITOL NP-30 | 5 | 5 | 5 | 5 | 5 | 5 | 5 | 5 | 5 |
| Polyquaternium-10¹ | 0.35 | — | 0.5 | 0.1 | 0.5 | — | 0.5 | — | 0.35 |
| Guar | 0.25 | — | — | 0.4 | — | 0.5 | — | 0.5 | — |
| Hydroxypropyltrimonium | | | | | | | | | |
| Chloride² | | | | | | | | | |
| Tricetylmonium Chloride | — | — | — | — | — | — | — | — | 0.5 |
| PEG7M³ | 0.05 | — | 0.1 | 0.1 | 0.1 | — | — | — | — |
| Zinc Pyrithione⁴ | 1 | 1 | 1 | 1 | 1 | 1 | 1 | 1 | — |
| Zinc Oxide | | — | — | — | — | — | — | 1.2 | — |
| Selenium Sulfide | | — | — | — | — | — | — | — | 1.0 |
| 1-decene homopolymer⁵ | 0.4 | — | 0.3 | 0.4 | 0.3 | 0.4 | 0.4 | 0.4 | — |
| Trimethylpropane Capyl | 0.2 | — | 0.1 | 0.1 | 0.1 | 0.1 | 0.1 | 0.1 | — |
| Caprylate⁶ | | | | | | | | | |
| Dimethicone⁷ | 3.25 | — | 1.3 | 1.3 | 1.3 | 2.5 | 2.2 | 2.2 | 0.63 |
| Ethylene Glycol Distearate | 1.0 | 1.0 | 1.5 | 1.5 | 1.5 | 1.5 | 1.5 | 1.5 | 3.0 |
| Cocamide MEA | 0.6 | 0.6 | 0.8 | 0.8 | 0.8 | 0.8 | 1.0 | 0.8 | 3.0 |
| Cetyl Alcohol | 0.6 | — | 0.9 | 0.9 | 0.9 | 0.9 | 0.9 | 0.9 | 0.42 |
| DMDM Hydantoin | — | — | — | — | — | — | — | — | 0.4 |
| Sodium Chloride | — | — | — | — | — | — | — | — | 0.3 |
| Stearyl Alcohol | — | — | — | — | — | — | — | — | 0.2 |
| Hydroxypropyl | — | — | — | — | — | — | — | — | 0.02 |
| Methylcellulose | | | | | | | | | |

| | |
|---|---|
| Water and minors | quantity sufficient |

¹UCARE Polymer LR400, available from Amerchol.
²Guar having a molecular weight of about 200,000, and having a charge density of about 0.71 meq/g, available from Aqualon.
³Polyox WSR N-750, available from Union Carbide.
⁴ZPT having an average particle size of about 2.5 μm, available from Arch/Olin.
⁵Puresyn 6, available from Mobil.
⁶Mobil P43, available from Mobil.
⁷Visasil 330,000 csk, available from General Electric Silicones.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Detailed Description of the Invention are, in relevant part, incorporated herein by reference; the citation of any document is not to be construed as an admission that it is prior art with respect to the present invention.

What is claimed:

1. A personal care composition comprising:
a) from about 5 to about 50 weight percent of a detersive surfactant,
b) from about 0.1 to about 4 weight percent of an anti-dandruff agent,
c) from about 0.1 to about 50 weight percent of an octylphenol ethoxylate, represented by the following formula 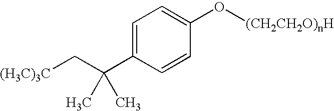
wherein n is a value of from about 3 to about 70, or a nonylphenol ethoxylate, represented by the following formula 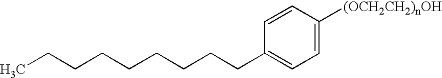
wherein n is a value of from about 4 to about 70, and
d) at least about 20 weight percent of water.

2. The composition of claim 1 wherein said anti-dandruff agent is a zinc salt of 1-hydroxy-2-pyridinethione.

3. The composition of claim 1, wherein said anti-dandruff agent is selenium sulfide.

4. The composition of claim 1, wherein said anti-dandruff agent is ketoconazole.

5. The composition of claim 1, comprising from about 0.3% to about 2% of said anti-dandruff agent.

6. The composition of claim 1, wherein said anti-dandruff agent is a particulate having an average particle size of about 2.5 μm.

7. The composition of claim 1, comprising from about 1% to about 20% of said octylphenol ethoxylate or nonylphenol ethoxylate.

8. The composition of claim 1, wherein n of said octylphenol exthoxylate is a value from about 5 to about 20.

9. The composition of claim 1, wherein n of said octylphenol ethoxylate is a value from about 5 to about 10.

10. The composition of claim 1, wherein n of said nonylphenol exthoxylate is a value from about 4 to about 30.

11. The composition of claim 1, wherein n of said nonylphenol exthoxylate is a value from about 7 to about 15.

12. The composition of claim 1, further comprising from about 0.05% to about 3%, by weight of the composition, of a cationic polymer.

13. The composition of claim 10, wherein said cationic polymer is a guar derivative.

14. The composition of claim 1, further comprising from about 0.01% to about 10%, by weight of the composition, of a silicone conditioning agent.

15. A method for providing cleansing and anti-dandruff efficacy comprising:
a) wetting a user's hair or skin with water;
b) applying an effective amount of said composition to hair or skin; and
c) rinsing said composition from hair or skin using water.
